(19) United States
(12) Patent Application Publication
(10) Pub. No.: US 2023/0241153 A1
(43) Pub. Date: Aug. 3, 2023

(54) METHODS AND COMPOSITIONS FOR UPREGULATING RNA INTERFERENCE AND ENHANCING GENE SILENCING

(71) Applicant: GOWEY RESEARCH GROUP, PLLC, Flagstaff, AZ (US)

(72) Inventor: Brandie Gowey, Flagstaff, AZ (US)

(21) Appl. No.: 18/164,368

(22) Filed: Feb. 3, 2023

Related U.S. Application Data
(63) Continuation-in-part of application No. PCT/US2021/044399, filed on Aug. 3, 2021.
(60) Provisional application No. 63/060,467, filed on Aug. 3, 2020.

(51) Int. Cl. A61K 38/01; A61K 33/04; A61K 31/714; A61K 33/06; A61K 38/06; A61K 36/185; A61K 38/16

(52) U.S. Cl. CPC A61K 38/011; A61K 33/04; A61K 31/714; A61K 33/06; A61K 38/063; A61K 36/185; A61K 38/162

(57) ABSTRACT

The fields of virology, oncology, and primary care need a drug that can upregulate expression of genes responsible for viral and abnormal cell clearance, such as p53 and those associated with or within the RNA interference (RNAi) process. It is well-known that cellular responses to viral genes and proteins, and indeed that of precancerous or cancerous cells, require the mechanistic efforts via p53 and the RNAi. Without the p53/RNAi activities, viral load and abnormal cell presence may remain or create disease processes and symptoms. The present invention features compositions (e.g., Sarravis® Core) and methods that allow for a decrease in viral and/or cancer cell expression through up regulation of RNAi, p53, and associated cofactors.

### CROSS-REFERENCES TO RELATED APPLICATIONS

This application is a continuation-in-part and claims benefit of International Application No. PCT/US2021/044399 filed Aug. 3, 2021, which claims priority to U.S. Provisional Application No. 63/060,467 filed Aug. 3, 2020, the specification(s) of which is/are incorporated herein in their entirety by reference.

### REFERENCE TO SEQUENCE LISTING

The contents of the electronic sequence listing (GOWB_20_01_PCT_CIP_Sequence_Listing.xml; Size: 22,132 bytes; and Date of Creation: Feb. 2, 2023) is herein incorporated by reference in its entirety.

### FIELD OF THE INVENTION

The present invention features compositions and methods that allow for a decrease in viral and/or cancer cell expression through gene silencing and upregulation of RNA interference coupled with drug medicaments that enhance viral/and/or cancer cell elimination.

### BACKGROUND OF THE INVENTION

RNA interference (RNAi) is a biological process in which RNA molecules are involved in sequence-specific suppression of gene expression. The RNAi pathway is found in many eukaryotes, and is initiated by the enzyme DICER. The DICER enzyme recognizes the presence of non-self genetic material and cleaves exogenous DNA and RNA, like a pair of scissors, into short double-stranded fragments. The double-stranded fragments are then unwound into two single-stranded pieces. One single-stranded fragment is incorporated into the RNA-induced silencing complex (RISC). Post-transcriptional gene silencing occurs when the single-strand fragment pairs with a complementary sequence in the DNA or RNA molecule and induces cleavage by the argonaute proteins (AGO; specifically AGO2), the catalytic component of the RISC. Additionally, the immune system recognizes these nucleotide fragments, and initiates a rise in cytokine expression upon recognition of non-self gene segments.

DICER and Argonaute proteins (AGO) are essential to cell maintenance, integrity of the genome, and apoptosis through coordination with p53 for said functions. Additionally, the aforementioned proteins (e.g., AGO and p53) are needed within the RNAi process for the detoxification of viral genes. Furthermore, viruses come with not just proteins, but many DNA and RNA components that can remain even if the protein is not present, making consistent gene silencing necessary: viral proteins may appear later due to transcription and translation and/or a lack of gene silencing. This places constant and considerable strains on the immune system to respond. As such, patients are at risk of immune exhaustion, a known phenomenon among those dealing with both chronic viral and abnormal/cancerous cell expressions. Hence, there is a need for treatments that can upregulate the expression of gene silencing aspects of the RNAi process, while simultaneously supporting clearance or inhibition of viral/abnormal cells.

### BRIEF SUMMARY OF THE INVENTION

It is an objective of the present invention to provide compositions (e.g., Sarravis® Core) and methods that allow for a decrease in viral and cancer cells and expression through simultaneous upregulation of RNAi and associated cofactors therein with inhibition/clearance of said cells, as specified in the independent claims. Embodiments of the invention are given in the dependent claims. Embodiments of the present invention can be freely combined with each other if they are not mutually exclusive.

Without wishing to limit the invention to any theory or mechanism, the compositions described herein can cause a rise in RNAi cofactors such as DICER and AGO2. Integrated viral and abnormal cell load is released as DICER/AGO2 mRNA count increases.

Without the RNAi activities, foreign DNA or RNA and abnormal cell presence may persist or create disease processes and symptoms. For example, without adequate levels of the DICER protein, non-self genetic material cannot be cleaved into smaller pieces for immune system and cytokine recognition. However, RNAi activities (and p53), may elicit detoxification symptoms related to the disease. For example, if a patient struggles with Epstein Barr, the patient may experience a rise in fatigue upon initial use of the Sarravis® Core-containing medicine (i.e. upon initial elevations in p53 and AGO2), but as viral load (or cancerous cell load) decreases, the symptoms resolve.

According to some embodiments, the present invention features a composition comprising about 0.1%-99% wt of an herbal preparation of a carnivorous plant, about 0.01%-95% wt of magnesium, glutathione, or both magnesium and glutathione, and about 0.01%-95% wt of a drug product. In some embodiments, the herbal preparation of the carnivorous plant is prepared from Sarracenia flava, Sarracenia purpurea, or mixed hybrids thereof. In other embodiments, the composition may further comprise zinc, chromium, selenium, B-vitamins, keratin, cannabidiol, tetrahydrocannabinol, or a combination thereof.

In some embodiments, the composition may be formulated for oral or topical administration, or for intramuscular, subcutaneous, or intravenous injections. Without wishing to limit the invention to any theory or mechanism, the composition may be effective for decreasing viral and/or cancer cell expression, enhancing gene silencing, enhancing p53 gene expression, upregulating RNA interference, or a combination thereof.

According to some embodiments, the present invention features a method of enhancing gene silencing in a subject in need thereof. The method may comprise administering to the subject a therapeutic amount of any of the compositions described herein.

According to other embodiments, the present invention features a method of treating a viral disease in a subject in need thereof. The method may comprise administering to the subject a therapeutic amount of any of the compositions described herein. In some embodiments, the method may further comprise testing the subject for a virus using polymerase chain reaction (PCR) or mRNA testing prior to administering the composition, and retesting the subject for the virus using PCR or mRNA testing after administering the composition. If the virus is still present in the subject, a result of the testing will come back positive and/or will indicate presence of m RNA for that particular virus. The viral disease may be Coronavirus Disease 2019 (COVID-19), a human papillomavirus (HPV) infection, a herpes simplex virus (HSV) infection, or mononucleosis caused by Epstein-Barr virus (EBV). In further embodiments, the composition may be combined with a viral protein for administration to the subject.

In some embodiments, the composition used in the methods described herein can be administered orally, topically, parenterally, intramuscularly, subcutaneously, or by intravenous injections.

According to other embodiments, the present invention features a composition for treating a human papillomavirus (HPV) or herpes simplex virus (HSV) infection. The composition may comprise about 0.1%-99% wt of an herbal preparation of a pitcher plant, about 0.01%-95% wt of magnesium, and about 0.01%-95% wt of valacyclovir. In other embodiments, the composition may further comprise about 0.01%-95% wt of a protease inhibitor, about 0.01%-95% wt a DNA/RNA polymerase inhibitor, or a combination thereof.

According to some other embodiments, the present invention features a composition comprising about 0.1%-99% wt of keratin and about 0.01%-95% wt of magnesium, glutathione, or both magnesium and glutathione. The keratin can be extracted from a carnivorous plant, such as pitcher plant, or produced synthetically. In some embodiments, the composition may further comprise zinc, chromium, selenium, B-vitamins, one or more drug products, cannabidiol, tetrahydrocannabinol, or a combination thereof. The composition can be formulated for oral, sublingual, or topical administration, or for intramuscular, subcutaneous or intravenous injections.

Keratins can transfect the nucleus and are able to carry bound cofactors (e.g., magnesium, selenium, or zinc) into the cell and to the nucleus. Additionally, cofactor vitamins and minerals are necessary for moderating inflammatory responses and DNA repair. Most, if not all, vitamins and minerals play roles in preventing inflammatory responses per immune system and endothelial cells. Without wishing to limit the invention to any theory or mechanism, the composition may be effective for decreasing viral and/or cancer cell expression, enhancing gene silencing, enhancing p53 gene expression, upregulating RNA interference, or a combination thereof.

In some embodiments, the drug products are combined with the compositions for enhanced outcomes for the patient, such as that of valacyclovir or protease inhibitors for antiviral effects, or cisplatin for oncologic effects. In some embodiments, the drug product comprises an antiviral drug, an anti-inflammatory drug, a cancer-modulating drug, an immune-modulating drug, a polymerase inhibitor, a protease inhibitor, or a combination thereof. Non-limiting examples of the drug product include acyclovir, valacyclovir, vidarabine, famciclovir, nitazoxanide, remdesivir, nirmatrelvir, ritonavir, molnupiravir, zanamivir, peramivir, baloxavir marboxil, oseltamivir phosphate, saquinavir, indinavir, nelfinavir, amprenavir, fosamprenavir, lopinavir, atazanavir, tipranavir, darunavir, asunaprevir, boceprevir, grazoprevir, glecaprevir, paritaprevir, simeprevir, telaprevir, ensitrelvir, cisplatin, mercaptopurine, carboplatin, paclitaxel, topotecan, vinblastine, gemcitabine docetaxel, doxorubicin, α-amanitin, mithramycin A, tenofovir, abacavir hemisulfate, delavirdine mesylate, azidothymidine, aphidicolin, entecavir, favipiravir, galidesivir dihydrochloride, BMS 986094, EIDD 1931, GS 441524, BMH 21, emetine dihydrochloride, rilpivirine, acetaminophen, cortisone, hydrocortisone, prednisone, indomethacin, ibuprofen, aspirin, celecoxib, or naproxen, or a combination thereof.

In some other embodiments, the present invention features a composition comprising keratin and one or more cofactors. In one embodiment, the composition comprises 0.1%-99% volume (vol) or 0.1%-99% weight (wt) of keratin and 0.01%-95% vol or 0.01%-95% wt of one or more cofactors. The keratin may comprise keratin I, keratin II, cytoskeletal types of keratin or a combination thereof. In some embodiments, the keratin is extracted from a pitcher plant or synthetically derived, which can also be successfully blended with one or more cofactors.

In some embodiments, the present invention may feature a composition for enhancing gene silencing, the composition comprising keratin and glutathione and/or magnesium. In other embodiments, the present invention features a composition enhancing p53 gene expression, the composition comprising keratin and glutathione and/or magnesium. In further embodiments, the present invention features a composition comprising keratin, magnesium, B vitamins, and glutathione.

In some embodiments, the present invention features a composition comprising zinc and one or more subunits of a coronavirus spike protein. In some embodiments, the one or more subunits of the coronavirus spike protein comprise a spike protein subunit 1 (S1), a spike protein subunit 2 (S2), or a combination thereof.

In other embodiments, the present invention may feature a method of enhancing gene silencing and/or enhancing the expression of p53 in a subject in need thereof. In some embodiments, the method comprises administering a therapeutic amount of a composition comprising keratin and one or more cofactors, to the subject.

In some embodiments, the present invention features a method decreasing viral and/or cancer cell expression in a subject in need thereof. In other embodiments, the present invention features a method of enhancing gene silencing in a subject in need thereof. In further embodiments, the present invention features a method of enhancing the expression of p53 in a subject in need thereof. In some embodiments, the methods described herein comprise administering a therapeutic amount of a composition comprising 0.1%-99% vol and/or w of keratin and 0.01%-95% vol and/or w of one or more cofactors, to the subject.

In some embodiments, the present invention features a method of treating a disease caused by a virus in a subject in need thereof. In some embodiments, the method comprises administering a therapeutically effective amount of a composition comprising keratin and one or more cofactors to the subject. In some embodiments, the composition may be combined with a viral protein.

Without wishing to limit the invention to any theory or mechanism, it is believed that the technical feature of the present invention advantageously provides for the enhancement of viral and oncologic therapies, due to additional rises in p53 with keratin/cofactor medicament administration, making abnormal and viral-infected cells available for immune and drug recognition (due to shifts in DICER/AGO2). None of the presently known prior references or work has the unique inventive technical feature of the present invention.

Viruses have abilities to integrate into the host genome and cellular components, (e.g., endoplasmic reticulum). The inventor has found that integration of the virus into the host genome limits PCR identification, generally missing the diagnosis of the viral presence because it has been identified by the inventor that PCR can only test for unbound DNA and RNA nucleotide sequences. HPV, Epstein Barr, and Coronaviruses all carry evolutionary strategies that allow them to hide within cells and produce genes. Thus, PCR is not properly identifying viral load and the viruses are still in the tissues. This poses a challenge to medicine because regardless of the immune response, the virus can still control the host cellular and gene expression through their genes (e.g., mRNA and miRNA). Hence, it is not the presence of the virus itself that yields disease, but rather the level of the viral mRNA itself.

Moreover, the prior references teach away from the present invention. For example, virology and oncology practices seek to suppress the presence of viral proteins as opposed to m RNA of the virus itself (or cancerous cell expression). Additionally, current oncology practices use chemotherapy and radiation to suppress DNA repair and proteins such as p53. Moreover, drug products are being sought out to suppress keratin proteins, so as to inhibit metastasis. The prevailing concept is that if there is an immune response to a particular viral protein (or cancerous cell expression), that cure is obtained. However, viruses (and to that end, cancerous cells) are far too complex to visualize from such a simple concept. This may be why there is a constant need for new vaccines for the same virus, i.e. the influenza virus, or new chemotherapeutics for the same cancer types.

In addition, the inventive technical features of the present invention contributed to a surprising result. For example, the combination of Sarravis® Core with cofactors and further combined with medicaments caused a suppression of cancerous cell development, as shown by cancer cell stabilization (Table 4). Additionally, it was discovered that Sarravis® Core combined with specific cofactor nutrients and administered as a medicament specifically directed to tissues affected by viral genes or precancerous/cancerous cells yielded surprising results. For example, an increase in gene expression of p53, DICER and Argonaute 2 (AGO2) was obtained. Equally as interesting and surprising is that the rise in DICER and AGO2 demonstrated an appearance of viral load in tissues previously diagnosed as within normal limits. For example, cervical cell swabs initially reported as being negative for HPV showed positive HPV results upon administration of Sarravis® Core medicaments (Table 2).

Furthermore, it was discovered that gene silencing alone was often not enough to eliminate viral presence/precancerous/cancerous cells. Additional medicaments were necessary in combination with the Sarravis® Core for favorable patient outcomes, such as elimination of precancerous cells.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B show data that demonstrates the impact on a patient of pre- (FIG. 1A) and post- (FIG. 1B) use of a topical keratin and magnesium formulation according to an embodiment of the present invention.

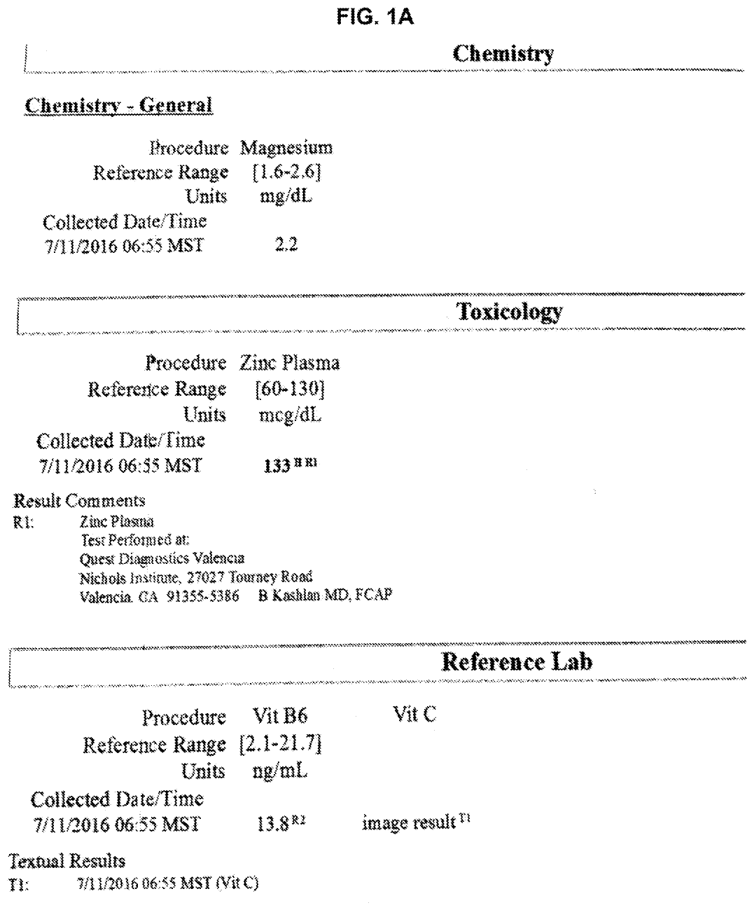

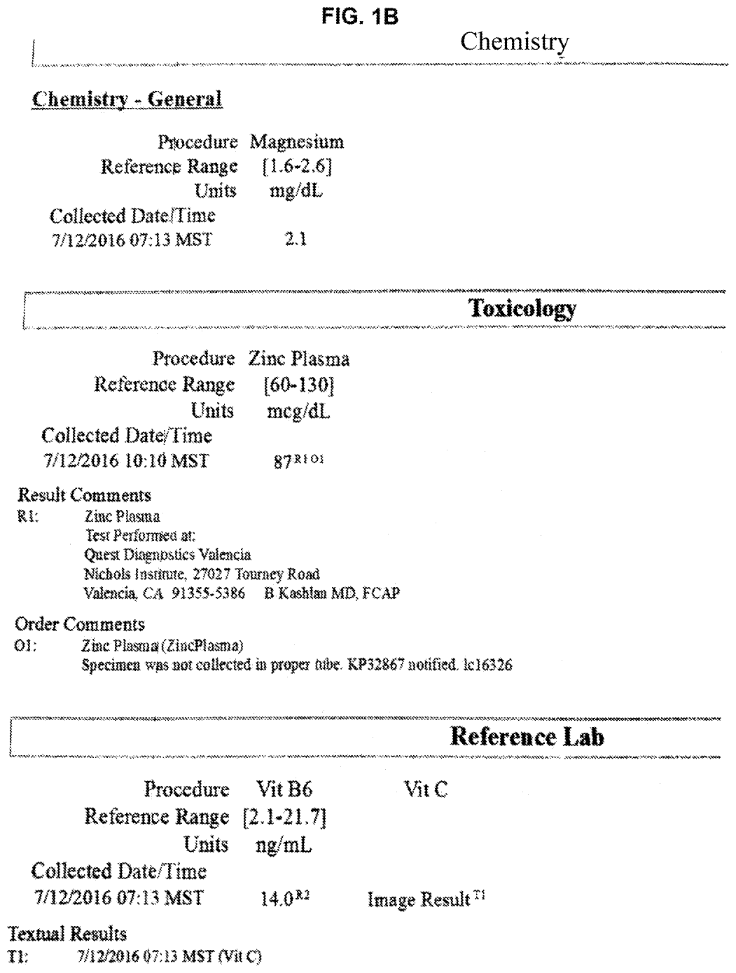

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”)

As known to one of ordinary skill in the art, a “carnivorous plant” is a predatory plant that obtains its nutrients by trapping and killing prey (International Carnivorous Plant Society, www.carnivorousplants.org). A carnivorous plant has the following features: 1. the plant captures and kills prey; 2. the plant has some mechanism to digest the prey; and 3. the plant absorbs the nutrients from the prey. Some non-limiting examples of carnivorous plants include species from the genus Sarracenia, Nepenthes, Dionaea, Utricularia, Hefiamphora, Cephalotus, and Drosera. The preferred carnivorous plants of the present invention are pitcher plants, namely, Sarracenia flava and Sarracenia purpurea. Since these plants can readily interbreed, the carnivorous plants can include hybrids thereof.

As defined herein, the terms “treating” or “treatment” of a condition includes: (1) preventing the condition, i.e., causing the clinical symptoms of the condition not to develop in a mammal that may be exposed to or predisposed to the condition but does not yet experience or display symptoms of the condition; (2) inhibiting the condition, i.e., arresting or reducing the development of the condition or its clinical symptoms; or (3) ameliorating or relieving the condition, i.e., causing regression of the condition or its clinical symptoms. As used herein, the terms “treat” or “treatment” refer to both therapeutic treatment or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the patient may be identified (e.g., diagnosed) as one suffering from the disease or condition prior to administration of the composition of the invention.

A “therapeutically effective amount” refers to an amount that is sufficient to achieve the desired therapeutic result or to have an effect on undesired symptoms but is generally insufficient to cause intolerable adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, “clinical improvement” may refer to a noticeable reduction in the symptoms of a disorder, or cessation thereof.

As used herein, the terms “administering” or “administer” is defined as the introduction of a substance (composition) into cells in vitro or into the body of an individual in vivo and includes topical, oral, nasal, ocular, rectal, vaginal and parenteral routes. The composition of the present invention may be administered via any route of administration including, but not limited to orally, sublingually, parenterally (e.g., intravenously and subcutaneously), by intramuscular injection, topically (including ophthalmically, vaginally, rectally, intranasally), by intraperitoneal injection, intrathecally, transderm ally, extracorporeally, intradermally or the like.

For example, the composition can be administered by needle injections into the subcutaneous tissue so as to up-regulate sulfhydryl oxidase, a key enzyme involved in building of disulfide bonds. Disulfide bonds are essential for activation of p53 and DNA repair processes.

The disclosed compounds can be administered topically, orally, intravenously, intraperitoneally, intramuscularly, subcutaneously, intracavity, transdermally, sublingually or through buccal delivery.

A composition can also be administered by buccal delivery or by sublingual delivery. As used herein “buccal delivery” may refer to a method of administration in which the compound is delivered through the mucosal membranes lining the cheeks. In some embodiment, for a buccal delivery the composition is placed between the gum and the cheek of a patient. As used herein “sublingual delivery” may refer to a method of administration in which the compound is delivered through the mucosal membrane under the tongue. In some embodiments, for a sublingual delivery the composition is administered under the tongue of a patient.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, for example, U.S. Pat. No. 3,610,795, which is incorporated by reference herein.

As used herein, the term “extract” is defined as a separation of the beneficial (medicinal) components of an herb from the fibrous, less useful part of the plant. Extracts can be in a liquid, gel, or powdered form.

As used herein, the term “infuse” is defined as a procedure of withdrawing nutritive compounds of an herb into a medium, and allowing them to linger in the medium for a period of time to allow for the transfer of herbal extracts into the medium. An “infused solution” is the resulting solution with the nutritive compounds.

As used herein, an “herbal preparation” or a “plant preparation” may be an extract, tincture, or infused solution made or prepared from an herb or plant. The herbal preparation contains active components from the herb or plant. For example, the herbal preparation of a pitcher plant may be a pitcher plant extract, tincture, or infused solution. Another example of an herbal preparation is a curcumin preparation derived from turmeric, or a curcumin tincture, extract or infused solution. An Orchidaceae plant preparation may be an orchid extract, tincture, or infused solution derived from any Orchidaceae species. A Lilium plant preparation may be a lily extract, tincture, or infused solution derived from any Lilium species, such as a lily root extract prepared from Lilium candidum. A Rosa plant preparation may be a rose extract, tincture, or infused solution derived from any Rosa species, such as absolute rose oil prepared from Rosa damascena. A hemp plant preparation may be an extract, tincture, or infused solution containing cannabidiol (CBD), such as CBD oil.

In some embodiments, a pitcher plant component is extracted from carnivorous plants. In a non-limiting embodiment, the carnivorous plants are pitcher plants, namely, Sarracenia flava and Sarracenia purpurea. Since these plants can readily interbreed, the carnivorous plants can include hybrids thereof. As used herein, a “pitcher plant component”, or alternatively, an “active component” is defined as the beneficial (medicinal) plant parts/material of pitcher plant. For instance, the herbal preparation of the pitcher plant may be oil infused with the pitcher plant active component. The pitcher plant active component may comprise one or more compounds.

As a non-limiting example of extracting the active components from the pitcher plant, in some embodiments, the pitcher plant is cut into small pieces, pulverized, mashed, or chopped. The pitcher plant pieces are placed in a non-reactive storage container, such as glass or plastic. A required amount of liquid, such as water, alcohol, or vinegar, is added to the storage container. The mixture is set aside and allowed to incubate for a period of time. The pitcher plant's active components transfer from the plant material into the liquid. After the incubation period is over, any plant material solids are separated from the liquid. The resulting liquid is the pitcher plant tincture. In another embodiment, a pitcher plant extract is prepared by dehydrating the pitcher plant material and pulverizing or grinding the plant material into a powder.

As used herein, the terms “% wt” and “% w/v” can be used interchangeably and are defined as a % concentration of unit weight or mass to unit volume. For example, a % w/v may refer to a concentration in g/ml.

As used herein, the terms “% vol”, “% vol/vol” and “% v/v” can be used interchangeably and refer to a volume percentage of a component relative to the total volume of the solution or mixture. For example, 5% vol of component A may refer to 5 ml of component A to 100 ml of total volume of the mixture.

As used herein, the term “supplement” is generally understood to include, but is not limited to, vitamins, minerals, fiber, fatty acids, amino acids, and amine derivatives.

As used herein, the term “minerals” may be categorized into two kinds of minerals: macrominerals and trace minerals. Macrominerals include, but are not limited to, calcium, phosphorus, magnesium, sodium, potassium, chloride and sulfur. Trace minerals include, but are not limited to, iron, manganese, copper, iodine, zinc, cobalt, fluoride and selenium.

Examples of vitamins include, but are not limited to, retinoic acid (Vitamin A), Vitamin B3 in the form of niacin (nicotinic acid), niacinamide (nicotinamide) or inositol hexanicotinate, folic acid or folate, vitamin B-complex, vitamin C, vitamin D, vitamin E, and vitamin K. Non-limiting examples of fatty acids include phosphocholine, phosphatidylcholine, and phosphatidylserine.

Non-limiting examples of amino acids include cysteine and arginine, such as L-cysteine and L-arginine. Examples of amine derivatives include, but are not limited to, glucosamine.

For example, vitamin C and/or vitamin C derivatives include fatty acid esters of ascorbic acid, particularly ascorbyl palmitate. As another example, vitamin E and/or derivatives of vitamin E include tocotrienol and/or tocotrienol derivatives.

In some embodiments, examples of pharmaceutically acceptable salts of magnesium that may be used in the topical compositions describe herein include, but are not limited to, magnesium oxide, magnesium carbonate, magnesium chloride, magnesium sulfate, magnesium phosphate, magnesium bicarbonate, magnesium glycinate, magnesium aspartate, magnesium glutamate, magnesium adipate, magnesium citrate, magnesium orotate, magnesium taurate, magnesium lysinate, and the like. Examples of pharmaceutically acceptable salts of zinc that may be used in the topical compositions described herein include, but are not limited to, zinc chloride, zinc oxide, zinc sulfate, and the like. Examples of pharmaceutically acceptable salts of calcium that may be used in the topical compositions described herein include, but are not limited to, calcium acetate, calcium carbonate, calcium chloride, calcium citrate, and calcium gluconate.

In other embodiments, the selenium mineral may be in an organic or inorganic form. For example, an organic form of selenium that may be used in accordance with the present invention is selenomethionine. Inorganic forms of selenium include sodium selenite and sodium selenate.

Any of the minerals disclosed herein may be used in the form of pharmaceutically acceptable salts. As used herein, “pharmaceutically acceptable” is meant that which is useful for the preparation of a pharmaceutical composition and is generally safe, non-toxic and neither biologically nor otherwise undesirable and which is acceptable for veterinary use as well as in human pharmaceutics.

By “pharmaceutically acceptable salts” of a compound is meant salts which are pharmaceutically acceptable as defined herein and which have the desired pharmacological action of the parent compound. Such salts comprise useful salts are acid addition salts, which are formed by pharmaceutically acceptable free acids. The acid addition salts are obtained from inorganic acid, such as hydrochloric acid, nitric acid, phosphoric acid, sulfuric acid, hydrobromic acid, hydroiodic acid, nitrous acid or phosphorous acid and the like; or formed with pharmaceutically acceptable organic acids, such as aliphatic mono- and dicarboxylates, phenyl-substituted alkanoates, hydroxyalkanoates, alkanedioates, aromatic acids, aliphatic and aromatic sulphonic acids, acetic acid, benzenesulfonic acid, benzoic acid, camphorsulfonic acid, citric acid, ethane-sulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like. Such pharmaceutically nontoxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitro benzoate, hydroxybenzoate, methoxybenzoate, phthalate, terephthalate, benzene sulfonate, toluene sulfonate, chlorobenzene sulfonate, xylenesulfonate, phenyl acetate, phenylpropionate, phenylbutyrate, citrate, lactate, hydroxybutyrate, glycolate, malate, tartrate, methane sulfonate, propane sulfonate, naphthalene-1-sulfonate, naphthalene-2-sulfonate or mandelate.

In other embodiments, the pharmaceutically acceptable salts may comprise the addition salts of pharmaceutically acceptable bases formed when an acid proton contained in the parent compound is either replaced by a metal ion e.g. an alkaline metal ion, an alkaline-earth metal ion or aluminium ion; or coordinated with a pharmaceutically acceptable organic or inorganic base. Acceptable organic bases include diethanolamine, ethanolamine, N-methylglucamine, triethanolamine, tromethamine and the like. Acceptable inorganic bases include aluminium hydroxide, calcium hydroxide, potassium hydroxide, sodium carbonate and sodium hydroxide.

In some embodiments, the compositions described herein may include bases that can encourage timed release. Non-limiting examples of such bases include hydrogels, micelles, vesicles, nanoparticles, hydroxypropyl methylcellulose (HPMC), poly(ethylene glycol) (PEG), polylactides (PLA), polyglycolides (PGA), poly(lactide-co-glycolides) (PLGA), polyanhydrides, and polyorthoesters.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

As used herein, “Sarravis® Core” refers to a composition comprising pitcher plant extracts with one or more cofactors. A non-limiting example of Sarravis® Core is a composition comprising pitcher plant extracts with magnesium. Another non-limiting example of Sarravis® Core is a composition comprising pitcher plant extracts with gluthathione. Yet another non-limiting example of Sarravis® Core is a composition comprising pitcher plant extracts with magnesium and gluthathione.

As used herein, the terms “those defined above” and “those defined herein” when referring to a variable incorporates by reference the broad definition of the variable as well as any narrow and/or preferred definitions, if any.

According to some embodiments, the present invention features a composition comprising about 0.1%-99% wt of an herbal preparation of a carnivorous plant, about 0.01%-95% wt of magnesium, glutathione, or both magnesium and glutathione, and about 0.01%-95% wt of a drug product. In some embodiments, the herbal preparation of the carnivorous plant is prepared from Sarracenia flava, Sarracenia purpurea, or mixed hybrids thereof. In other embodiments, the composition may further comprise zinc, chromium, selenium, B-vitamins, keratin, cannabidiol, tetrahydrocannabinol, or a combination thereof.

In some embodiments, the composition may be formulated for oral or topical administration, or for intramuscular, subcutaneous, or intravenous injections. Without wishing to limit the invention to any theory or mechanism, the composition may be effective for decreasing viral and/or cancer cell expression, enhancing gene silencing, enhancing p53 gene expression, upregulating RNA interference, or a combination thereof.

In some embodiments, the present invention features a method of enhancing gene silencing in a subject in need thereof. The method may comprise administering to the subject a therapeutic amount of any of the compositions described herein.

In other embodiments, the present invention features a method of treating a viral disease in a subject in need thereof. The method may comprise administering to the subject a therapeutic amount of any of the compositions described herein. In some embodiments, the method may further comprise testing the subject for a virus using polymerase chain reaction (PCR) or mRNA testing prior to administering the composition, and retesting the subject for the virus using PCR or mRNA testing after administering the composition. If the virus is still present in the subject, a result of the testing will come back positive and/or will indicate presence of mRNA for that particular virus. The viral disease may be Coronavirus Disease 2019 (COVID-19), a human papillomavirus (HPV) infection, a herpes simplex virus (HSV) infection, or mononucleosis caused by Epstein-Barr virus (EBV). In further embodiments, the composition may be combined with a viral protein for administration to the subject.

In some embodiments, the composition used in the methods described herein can be administered orally, topically, parenterally, intramuscularly, subcutaneously, or by intravenous injections.

According to other embodiments, the present invention features a composition for treating a human papillomavirus (HPV) or herpes simplex virus (HSV) infection. The composition may comprise about 0.1%-99% wt of an herbal preparation of a pitcher plant, about 0.01%-95% wt of magnesium, and about 0.01%-95% wt of valacyclovir. In other embodiments, the composition may further comprise about 0.01%-95% wt of a protease inhibitor, about 0.01%-95% wt a DNA/RNA polymerase inhibitor, or a combination thereof.

According to some other embodiments, the present invention features a composition comprising about 0.1%-99% wt of keratin and about 0.01%-95% wt of magnesium, glutathione, or both magnesium and glutathione. The keratin can be extracted from a carnivorous plant, such as pitcher plant, or produced synthetically. In some embodiments, the composition may further comprise zinc, chromium, selenium, B-vitamins, one or more drug products, cannabidiol, tetrahydrocannabinol, or a combination thereof. The composition can be formulated for oral, sublingual, or topical administration, or for intramuscular, subcutaneous or intravenous injections.

In some embodiments, the present invention features a composition comprising keratin and one or more cofactors. In some embodiments, the composition comprises about 0.1%-99% wt of keratin and about 0.01%-95% wt of one or more cofactors. In other embodiments, the composition comprises about 0.1%-99% wt of keratin and about 0.01%-50% wt of one or more cofactors. In some embodiments, the composition comprises about 0.1%-99% vol of keratin and about 0.01%-95% vol of one or more cofactors. In some embodiments, the composition comprises about 0.1%-99% vol of keratin and about 0.01%-50% vol of one or more cofactors.

In some embodiments, the compositions described herein can decrease viral and/or cancer cell expression. In some embodiments, the compositions described herein can enhance gene silencing. In other embodiments, the compositions described herein can enhance p53 gene expression. In some embodiments, the compositions described herein can enhance RNA interference (RNAi; i.e., a composition for enhancing gene silencing).

In some embodiments, the keratin is extracted from a source to form a keratin extract. In some embodiments, the keratin may be extracted from carnivorous plants. In some embodiments, the keratin may be extracted from a pitcher plant. For example, in one embodiment, the pitcher plant may be Sarracenia flava, Sarracenia purpurea, or hybrids thereof. In alternative embodiments, the keratin is synthesized. In further embodiments, the keratin is in a pre-existing form.

In some embodiments, the keratin may comprise keratin I, keratin II, cytoskeletal types of keratin or a combination thereof. In some embodiments, the keratin may be in a liquid form. In other embodiments, the keratin may be in a solid form (e.g., a powder form). Without wishing to limit the invention to any theory or mechanism, the keratin help move the entire molecule towards the nucleus, e.g. nuclear transfection.

According to some embodiments, the compositions described herein may be combined with additional cofactors, such as other herbal extracts, drugs, vitamins, minerals, essential fatty acids, amino acids, and amine derivatives.

In some embodiments, the compositions described herein may comprise the herbal preparation of a carnivorous plant at a range of about 0.1-99% wt or vol of the composition, or about 50-99% wt or vol of the composition, or about 0.1-50% wt or vol of the composition, or any range therein. In one embodiment, the composition may further comprise one or more cofactors, at a range of about 0.1-95% wt or vol of the composition. In another embodiment, the composition may further comprise one or more cofactors, at a range of about 0.1-50% wt or vol of the composition. In one embodiment, the composition may further comprise one or more cofactors, at a range of about 0.1-25% wt or vol of the composition.

In other embodiments, the composition may comprise the herbal preparation of a carnivorous plant at a range of about 0.1% to 99% vol (or % wt) of the composition, one or more vitamins at a range of about 0.001% to 25% vol (or % wt) of the composition, one or more minerals at a range of about 0.001% to 25% vol (or % wt) of the composition, and one or more amino acids at a range of about 0.001% to 25% vol (or % wt) of the composition. In other embodiments, the composition may comprise the herbal preparation of a carnivorous plant at a range of about 0.1% to 99% vol (or % wt) of the composition, one or more vitamins at a range of about 0.001% to 50% vol (or % wt) of the composition, one or more minerals at a range of about 0.001% to 50% vol (or % wt) of the composition, and one or more amino acids at a range of about 0.001% to 50% vol (or % wt) of the composition. In further embodiments, the composition may comprise the herbal preparation of a carnivorous plant at a range of about 0.1% to 99% vol of the composition, one or more vitamins at a range of about 0.001% to 95% vol (or % wt) of the composition, one or more minerals at a range of about 0.001% to 95% vol (or % wt) of the composition, and one or more amino acids at a range of about 0.001% to 95% vol (or % wt) of the composition.

In other embodiments, the compositions described herein may comprise the keratin at a range of about 0.1-99% wt or vol of the composition, or about 50-99% wt or vol of the composition, or about 0.1-50% wt or vol of the composition, or any range therein. In one embodiment, the composition may further comprise one or more cofactors, at a range of about 0.1-95% wt of the composition. In another embodiment, the composition may further comprise one or more cofactors, at a range of about 0.1-50% wt of the composition. In one embodiment, the composition may further comprise one or more cofactors, at a range of about 0.1-25% wt of the composition.

In some embodiments, the composition may comprise a keratin at a range of about 0.1% to 99% vol (or % wt) of the composition, one or more vitamins at a range of about 0.001% to 25% vol (or % wt) of the composition, one or more minerals at a range of about 0.001% to 25% vol (or % wt) of the composition, and one or more amino acids at a range of about 0.001% to 25% vol (or % wt) of the composition. In other embodiments, the composition may comprise a keratin at a range of about 0.1% to 99% vol (or % wt) of the composition, one or more vitamins at a range of about 0.001% to 50% vol (or % wt) of the composition, one or more minerals at a range of about 0.001% to 50% vol (or % wt) of the composition, and one or more amino acids at a range of about 0.001% to 50% vol (or % wt) of the composition. In further embodiments, the composition may comprise a keratin at a range of about 0.1% to 99% vol of the composition, one or more vitamins at a range of about 0.001% to 95% vol (or % wt) of the composition, one or more minerals at a range of about 0.001% to 95% vol (or % wt) of the composition, and one or more amino acids at a range of about 0.001% to 95% vol (or % wt) of the composition.

In some embodiments, the one or more cofactors are minerals. Non-limited examples of minerals that could be cofactors include but are not limited to calcium (Ca), phosphorus (P), magnesium (Mg), sodium (Na), potassium (K), chloride (CI), sulfur (S), iron (Fe), manganese (Mn), copper (Cu), iodine (I), zinc (Zn), cobalt (Co), fluoride (F), chromium (Cr), selenium (Se) or a combination thereof. In other embodiments, the cofactor is a vitamin. In some embodiments, the one or more cofactors are vitamins. Examples of vitamins include, but are not limited to, retinoic acid (Vitamin A), niacin, folic acid, B-vitamins (e.g., vitamin B-complex), vitamin C, vitamin D, vitamin E, and vitamin K. In some embodiments, the cofactor is glutathione. In further embodiments, the compositions described herein may be combined with other natural extracts including, but not limited to, rose extract or green tea extract.

In some embodiments, the composition may further comprise one or more of the following: aloe vera gel, glycerine, natural oils, an emulsifier, menthol crystals, berries, green tea extract, medicinal mushrooms, turmeric, ginger, viscum, burdock, devil's claw, boneset, valerian, skullcap, marshmallow root, mullein leaf, elecampane root, fennel seed, licorice root, old man's beard lichen, orange peel, osha root, wild cherry bark, propolis, ginkgo, poppy, polygonum, hops, passionflower, avena, and arnica.

In still other embodiments, the composition may further comprise one or more solutions such as, for example, a witch hazel solution, a lye solution, a salt solution, a carrier oil such as coconut oil, olive oil, castor oil, canola oil, sweet almond oil, apricot kernel oil, avocado oil, grapeseed oil, jojoba oil, sunflower oil, hemp seed oil, kukui nut oil, evening primrose oil, and a gelatin.

In some other embodiments, the composition may further comprise extracts from cannabis plants or hemp plants, such as cannabidiol (CBD) oil or hemp oil. In some embodiments, the composition may further comprise tetrahydrocannabinol (THC) and/or CBDs.

In other embodiments, cofactors associated with Sarravis® Core include, but are not limited to, those that would be relevant to the pathway desired to be treated. Examples include blending with glutathione to enhance gene silencing, or with magnesium to up regulate p53 via DNA stabilization (Table 4).

In other embodiments, cofactors associated with Sarravis® Core may include drug products that support or modulate the immune systems. In some embodiments, the drug products can enhance immune function and at the same time inhibit viral replication. Without wishing to limit the present invention to any theory or mechanism, it is believed that the drug products described herein as cofactors can support or modulate the ability of the immune system to eliminate cancerous, precancerous, and viral-infected cells once the RNAi processed have upregulated, thereby releasing said cells from the tissues and/or endothelium.

In some embodiments, the one or more cofactor is a drug product. In some embodiments, the drug product comprises antiviral drugs, DNA/RNA polymerase inhibitors, protease inhibitors, cancer-modulating drugs, immune-modulating drugs, or a combination thereof. Such examples of drug products that may be used as cofactors include, but are not limited to, antivirals such as acyclovir, valacyclovir, vidarabine, famciclovir, nitazoxanide, remdesivir, nirmatrelvir, ritonavir, molnupiravir, zanamivir, peramivir, baloxavir marboxil, or oseltamivir phosphate. Other antivirals known in the art may be used as a drug product in the compositions described herein.

In some embodiments, the drug products may include protease inhibitors such as, for example, ritonavir, saquinavir, indinavir, nelfinavir, amprenavir, fosamprenavir, lopinavir, atazanavir, tipranavir, and darunavir. Other examples of protease inhibitors that may be use include, but are not limited to, asunaprevir, boceprevir, grazoprevir, glecaprevir, paritaprevir, simeprevir, and telaprevir. In other embodiments, drug products that may be used as cofactors include SARS-COVID-2 protease inhibitors such as ensitrelvir and nirmatrelvir.

In some embodiments, drug products that are DNA/RNA polymerase inhibitors include, but are not limited to, remdesivir, acyclovir, α-amanitin, mithramycin A, tenofovir, abacavir hemisulfate, delavirdine mesylate, azidothymidine, aphidicolin, entecavir, BMS 986094, favipiravir, galidesivir dihydrochloride, EIDD 1931, GS 441524, BMH 21, emetine dihydrochloride, or rilpivirine.

In some embodiments, drug products that may be used as cofactors may include cancer-modulating drugs (or immunotherapeutics) including but not limited to cisplatin, mercaptopurine, carboplatin, paclitaxel, topotecan, vinblastine, gemcitabine docetaxel, or doxorubicin. In other embodiments, drug products that may be used as cofactors may include anti-inflammatory or immune-modulating drugs such as acetaminophen, corticosteroids such as cortisone, hydrocortisone, and prednisone, or NSAIDS such as indomethacin, ibuprofen, aspirin, celecoxib, and naproxen.

In one embodiment, compositions that include the drug products are combined together into one formulation. In some embodiments, compositions that include the drug products may comprise Sarravis® Core and the drug product combined together into one formulation. In other embodiments, compositions that include the drug products may comprise keratin, one or more cofactors, and the drug product combined together into one formulation. For example, the composition may comprise an injectable solution, a pill, or a topical formulation that has the Sarravis® Core and the drug product. As another example, the composition may comprise an injectable solution, a pill, or a topical formulation that has the keratin, one or more cofactors, and the drug product altogether.

In an alternative embodiment, compositions with the drug product are administered separately. In one embodiment, the composition may comprise Sarravis® Core and the drug product, which are administered separately. For example, Sarravis® Core may be administered by injection as an injectable solution and the drug product may be administered orally in pill form. As another example, Sarravis® Core may be administered topically and the drug product may be administered orally in pill form. As another example, Sarravis® Core may be administered orally and the drug product may be administered orally in pill form. As another example, Sarravis® Core may be administered orally and the drug product may be administered intravenously.

In another embodiment, the composition may comprise keratin, one or more cofactors, and the drug product, which are administered separately. For example, keratin and the one or more cofactors may be administered by injection as an injectable solution and the drug product may be administered orally in pill form. As another example, keratin and the one or more cofactors may be administered topically and the drug product may be administered orally in pill form. As another example, keratin and the one or more cofactors may be administered orally and the drug product may be administered orally in pill form. As another example, keratin and the one or more cofactors may be administered orally and the drug product may be administered intravenously.

In some embodiments, the compositions described herein may be used in a method of enhancing gene silencing in a subject in need thereof. In other embodiments, the compositions described herein may be used in a method enhancing the expression of p53 in a subject in need thereof. In some embodiments, the method comprises administering a therapeutic amount of a composition as described herein, to the subject.

In some embodiments, the present invention may feature a composition for enhancing gene silencing, the composition comprising keratin and glutathione. In some embodiments, the composition for enhancing gene silencing comprises 0.001%-99% vol and/or weight of keratin and 0.01%-95% vol and/or weight of glutathione. In other embodiments, the present invention features a composition for enhancing p53 gene expression, the composition comprising keratin and magnesium and/or glutathione. In some embodiments, the composition for enhancing p53 gene expression comprises 0.1%-99% vol and/or weight of keratin and 0.01%-95% vol and/or weight of magnesium and/or glutathione. In further embodiments, the present invention features a composition comprising: keratin, magnesium, B vitamins, bifidobacterium, and glutathione. In some embodiments, the composition comprises 0.001%-99% vol and/or weight of keratin, 0.01%-95% vol and/or weight of magnesium, 0.01%-50% vol and/or weight of B-vitamins, 0.01%-50% vol and/or weight of bifidobacterium, and 0.01%-95% vol and/or weight of glutathione. In other embodiments, the composition comprises 0.001%-99% vol and/or weight of keratin, 0.01%-95% vol and/or weight of magnesium, 0.01%-95% vol and/or weight of B-vitamins, 0.01%-95% vol and/or weight of bifidobacterium, and 0.01%-95% vol and/or weight of glutathione.

In some embodiments, the present invention features a composition comprising: zinc and one or more subunits of the coronavirus spike protein. In some embodiments, the composition comprises 0.01%-50% vol and/or weight of zinc and one or more subunits of the coronavirus spike protein. In some embodiments, the one or more subunits of the coronavirus spike protein comprise a spike protein subunit 1 (S1), a spike protein subunit 2 (S2), or a combination thereof

In further embodiments, compositions described herein (e.g., Sarravis® Core) may be blended with vaccine viral protein or mRNA for best results. For example, Sarravis® Core may be combined with the spike protein (e.g., the S1 subunit, the S2 subunit or a combination thereof; Table 1), an endoplasmic reticulum fusion protein, from the Coronavirus family of viruses. In some embodiments, the S1 subunit and/or the S2 subunit of the spike protein may be combined with Sarravis® Core with or without additional stabilizers (i.e., cofactors) such as zinc or chromium.

Without wishing to limit the present invention to any theories or mechanisms it is believed that because the endoplasmic reticulum is needed for protein synthesis, the immune responses may be strengthened with the upregulation of RNAi cofactors (e.g., DICER and AGO2). Additionally, in some embodiments, the S1 subunit and/or the S2 subunit of the spike protein may be utilized to stabilize the endoplasmic reticulum, in particular, if administered in vaccination form with a cofactor (e.g. zinc), with and without the Sarravis® Core medicaments.

In other embodiments, compositions described herein (e.g., Sarravis® Core) may be combined with other vaccine viral proteins such as proteins from Epstein Barr (e.g. latent membrane protein 1 (LMP1)) and HPV vaccines (e.g. the Major capsid protein L1 epitope of HPV; Table 1). For example, Epstein Barr protein vaccination medicines may be combined with the compositions described herein (e.g., Sarravis® Core and magnesium and/or glutathione and/or zinc).

In one embodiment, the present invention may comprise a vaccine comprising Sarravis® Core and an EBV viral protein. The EBV viral protein may comprise latent membrane protein 1 (LMP1).

In another embodiment, the present invention may comprise a vaccine comprising Sarravis® Core and an HPV viral protein. The HPV viral protein may comprise the Major capsid protein L1 epitope of HPV.

In yet another embodiment, the present invention may comprise a vaccine comprising Sarravis® Core and a herpes simplex virus (HSV) viral protein. The HSV viral protein may comprise any of the subunits of HSV.

In some embodiments, the aforementioned compositions are administered via injection. In other embodiments, the aforementioned compositions are administered sublingually. In other embodiments, the aforementioned compositions are administered topically. In other embodiments, the aforementioned compositions are administered orally or intranasally.

TABLE 1
Viral Protein Sequences.

| SEQ | | |
|---|---|---|
| ID NO: | Protein: | Sequence: |
| 1 | Spike | MFVFLVLLPLVSSQCVNLTTRTQLPPAYTNSFTRGVYYPDKVFRSSVLHST |
| | Glycoprotein | QDLFLPFFSNVTWFHAIHVSGTNGTKRFDNPVLPFNDGVYFASTEKSNIIR |
| | (signal | GWIFGTTLDSKTQSLLIVNNATNVVIKVCEFQFCNDPFLGVYYHKNNKSW |
| | sequence; S1 | MESEFRVYSSANNCTFEYVSQPFLMDLEGKQGNFKNLREFVFKNIDGYFK |
| | subunit; S2 | IYSKHTPINLVRDLPQGFSALEPLVDLPIGINITRFQTLLALHRSYLTPGDS |
| | subunit) | SSGWTAGAAAYYVGYLQPRTFLLKYNENGTITDAVDCALDPLSETKCTLKS |
| | | FTVEKGIYQTSNFRVQPTESIVRFPNITNLCPFGEVFNATRFASVYAWNRK |
| | | RISNCVADYSVLYNSASFSTFKCYGVSPTKLNDLCFTNVYADSFVIRGDEV |
| | | RQIAPGQTGKIADYNYKLPDDFTGCVIAWNSNNLDSKVGGNYNYLYRLFR |
| | | KSNLKPFERDISTEIYQAGSTPCNGVEGFNCYFPLQSYGFQPTNGVGYQP |
| | | YRVVVLSFELLHAPATVCGPKKSTNLVKNKCVNFNFNGLTGTGVLTESNKK |
| | | FLPFQQFGRDIADTTDAVRDPQTLEILDITPCSFGGVSVITPGTNTSNQVA |
| | | VLYQDVNCTEVPVAIHADQLTPTWRVYSTGSNVFQTRAGCLIGAEHVNNS |
| | | YECDIPIGAGICASYQTQTNSPRRARSVASQSWAYTMSLGAENSVAYSNNS |
| | | IAIPTNFTISVTTEILPVSMTKTSVDCTMYICGDSTECSNLLLQYGSFCTQL |
| | | NRALTGIAVEQDKNTQEVFAQVKQIYKTPPIKDFGGFNFSQILPDPSKPSK |
| | | RSFIEDLLFNKVTLADAGFIKQYGDCLGDIAARDLICAQKFNGLTVLPPLLT |
| | | DEMIAQYTSALLAGTITSGWTFGAGAALQIPFAMQMAYRFNGIGVTQNVLY |
| | | ENQKLIANQFNSAIGKIQDSLSSTASALGKLQDVVNQNAQALNTLVKQLSS |
| | | NFGAISSVLNDILSRLDKVEAEVQIDRLITGRLQSLQTYVTQQLIRAAEIR |
| | | ASANLAATKMSECVLGQSKRVDFCGKGYHLMSFPQSAPHGVVFLHVTYVPA |
| | | QEKNFTTAPAICHDGKAHFPREGVFVSNGTHWFVTQRNFYEPQIITTDNTF |
| | | VSGNCDVVIGIVNNTVYDPLQPELDSFKEELDKYFKNHTSPDVDLGDISGI |
| | | NASWNIQKEIDRLNEVAKNLNESLIDLQELGKYEQYIKWPWYIWLGFIAGL |
| | | IAIVMVTIMLCCMTSCCSCLKGCCSCGSCCKFDEDDSEPVLKGVKLHYT |
| 2 | Major Capsid | MSLWLPSEATVYLPPVPVSKVVSTDEYVARTNIYYHAGTSRLLAVGHPYF |
| | (L1) Protein | PIKKPNNNKILVPKVSGLQYRVFRIHLPDPNKFGFPDTSFYNPDTQRLVW |
| | of HPV type | ACVGVEVGRGQPLGVGISGHPLLNKLDDTENASAYAANAGVDNRECISMD |
| | 16 | YKQTQLCLIGCKPPIGEHWGKGSPCTNVAVNPGDCPPLELINTVIQDGDM |
| | | RVDTGFGAMDFTTLQANKSEVPLDICTSICKYPDYIKMVSEPYGDSLFFYL |
| | | REQMFVRHLFNRAGAVGENVPDDLYIKGSGSTANLASSNYFPTPSGSMV |
| | | TSDAQIFNKPYWLQRAQGHNNGICWGNQLFVTVVDTTRSTNMSLCAAISTS |
| | | ETTYKNTNFKEYLRHGEEYDLQFIFQLCKITLTADVMTYIHSMNSTILEDW |
| | | NFGLQPPPGGTLEDTYRFVTSQAIACQKHTPPAPKEDPLKKYTFWEVNLK |
| | | EKFSADLDQFPLGRKFLLQAGLKAKPKFTLGKRKATPTTSSTSTTAKRKKR |
| | | KL |
| 3 | Major Capsid | MCLYTRVLILHYHLLPLYGPLYHPRPLPLHSILVYMVHIIICGHYIILFLRN |
| | (L1) Protein | VNVFPIFLQMALWRPSDNTVYLPPPSVARVVNTDDYVTPTSIFYHAGSSRLL |
| | of HPV type | TVGNPYFRVPAGGGNKQDIPKVSAYQYRVFRVQLPDPNKFGLPDTSIYNPE |
| | 18 | TQRLVWACAGVEIGRGQPLGVGLSGHPFYNKLDDTESSHAATSNVSEDV |
| | | RDNVSVDYKQTQLCILGCAPAIGEHWAKGTACKSRPLSQGDCPPLELKNT |
| | | VLEDGDMVDTGYGAMDFSTLQDTKCEVPLDICQSICKYPDYLQMSADPY |
| | | GDSMFFCLRREQLFARHFWNRAGTMGDTVPQSLYIKGTGMPASPGSCV |
| | | YSPSPSGSIVTSDSQLFNKPYWLHKAQGHNNGVCWHNQLFVTVVDTTPS |
| | | TNLTICASTQSPVPGQYDATKFKQYSRHVEEYDLQFIFQLCTITLTADVMS |
| | | YIHSMNSSILEDWNFGVPPPPTTSLVDTYRFVQSVAITCQKDAAPAENKDP |
| | | YDKLKFWNVDLKEKFSLDLDQYPLGRKFLVQAGLRRKPTIGPRKRSAPSA |
| | | TTSSKPAKRVRVRARK |
| 4 | Major Capsid | MWRPSDSTVYVPPPNPVSKVVATDAYVTRTNIFYHASSSRLLAVGHPYFS |
| | (L1) Protein | IKRANKTVVPKVSGYQYRVFKWLPDPNKFALPDSSLFDPTTQRLVWACT |
| | of HPV type 6 | GLEVGRGQPLGVGVSGHPFLNKYDDVENSGSGGNPGQDNRVNVGMDYK |
| | | QTQLCMVGCAPPLGEHWGKGKQCTNTPVQAGDCPPLELITSVIQDGDMV |
| | | DTGFGAMNFADLQTNKSDVPIDICGTTCKYPDYLQMAADPYGDRLFFFL |
| | | RKEQMFARHFFNRAGEVGEPVPDTLIIKGSGNRTSVGSSIYVNTPSGSLV |
| | | SSEAQLFNKPYWLQKAQGHNNGICWGNQLFVTWDTTRSTNMTLCASVAT |
| | | SSTYTNSDYKEYMRHVEEYDLQFIFQLCSITLSAEVMAYIHTMNPSVLED |
| | | WNFGLSPPPNGTLEDTYRYVQSQAITCQKPTPEKEKPDPYKNLSFWEVN |
| | | LKEKFSSELDQYPLGRKFLLQSGYRGRSSIRTGVKRPAVSKASAAPKRKR |
| | | AKTKR |
| 5 | Major Capsid | MWRPSDSTVYVPPPNPVSKVVATDAYVKRTNIFYHASSSRLLAVGHPYYS |
| | (L1) Protein | IKKVNKTVVPKVSGYQYRVFKWLPDPNKFALPDSSLFDPTTQRLVWACT |
| | of HPV type | GLEVGRGQPLGVGVSGHPLLNKYDDVENSGGYGGNPGQDNRVNVGMD |
| | 11 | YKQTQLCMVGCAPPLGEHWGKGTQCSNTSVQNGDCPPLELITSVIQDGD |
| | | MVDTGFGAMNFADLQTNKSDVPLDICGTVCKYPDYLQMAADPYGDRLFF |
| | | YLRKEQMFARHFFNRAGTVGEPVPDDLLVKGGNNRSSVASSIYVHTPSG |
| | | SLVSSEAQLFNKPYWLQKAQGHNNGICWGNHLFVTVVDTTRSTNMTLCA |
| | | SVSKSATYTNSDYKEYMRHVEEFDLQFIFQLCSITLSAEVMAYIHTMNPSV |
| | | LEDWNFGLSPPPNGTLEDTYRYVQSQAITCQKPTPEKEKQDPYKDMSFW |
| | | EVNLKEKFSSELDQFPLGRKFLLQSGYRGRTSARTGIKRPAVSKPSTAPK |
| | | RKRTKTKK |
| 6 | Major Capsid | MSLWRPSEATVYLPPVPVSKWVSTDEYVTRTNIYYHAGSARLLTVGHPYY |
| | (L1) Protein | SIPKSDNPKKIWPKVSGLQYRVFRVRLPDPNKFGFPDTSFYNPETQRLV |
| | of HPV type | WACVGLEVGRGQPLGVGISGHPLLNKFDDTENSNRYAGGPGTDNRECIS |
| | 31 | MDYKQTQLCLLGCKPPIGEHWGKGSPCSNNAITPGDCPPLELKNSVIQDG |
| | | DMVDTGFGAMDFTALQDTKSNVPLDICNSICKYPDYLKMVAEPYGDTLFF |
| | | YLRREQMFVRHFFNRSGTVGESVPTDLYIKGSGSTATLANSTYFPTPSGS |
| | | MVTSDAQIFNKPYWMQRAQGHNNGICWGNQLFVTVVDTTRSTNMSVCAAI |
| | | ANSDTTFKSSNFKEYLRHGEEFDLQFIFQLCKITLSADIMTYIHSMNPAIL |
| | | EDWNFGLTTPPSGSLEDTYRFVTSQAITCQKTAPQKPKEDPFKDYVFWEV |
| | | NLKEKFSADLDQFPLGRKFLLQAGYRARPKFKAGKRSAPSASTTTPAKRK |
| | | KTKK |
| 7 | Major Capsid | MSVWRPSEATVYLPPVPVSKVVSTDEYVSRTSIYYYAGSSRLLAVGHPYF |
| | (L1) Protein | SIKNPTNAKKLLVPKVSGLQYRVFRVRLPDPNKFGFPDTSFYNPDTQRLV |
| | of HPV type | WACVGLEIGRGQPLGVGISGHPLLNKFDDTETGNKYPGQPGADNRECLS |
| | 33 | MDYKQTQLCLLGCKPPTGEHWGKGVACTNAAPANDCPPLELINTIIEDGD |
| | | MVDTGFGCMDFKTLQANKSDVPIDICGSTCKYPDYLKMTSEPYGDSLFFF |
| | | LRREQMFVRHFFNRAGTLGEAVPDDLYIKGSGTTASIQSSAFFPTPSGSM |
| | | VTSESQLFNKPYWLQRAQGHNNGICWGNQVFVTVVDTTRSTNMTLCTQV |
| | | TSDSTYKNENFKEYIRHVEEYDLQFVFQLCKVTLTAEVMTYIHAMNPDILE |
| | | DWQFGLTPPPSASLQDTYRFVTSQAITCQKTVPPKEKEDPLGKYTFWEVD |
| | | LKEKFSADLDQFPLGRKFLLQAGLKAKPKLKRAAPTSTRTSSAKRKKVKK |
| 8 | Major Capsid | MAHNIIYGHGIIIFLKNVNVFPIFLQMALWRPSDSTVYLPPPSVARVVSTDD |
| | (L1) Protein | YVSRTSIFYHAGSSRLLTVGNPYFRWPNGAGNKQAVPKVSAYQYRVFRV |
| | of HPV type | ALPDPNKFGLPDSTIYNPETQRLVWACVGMEIGRGQPLGIGLSGHPFYNK |
| | 45 | LDDTESAHAATAVITQDVRDNVSVDYKQTQLCILGCVPAIGEHWAKGTLC |
| | | KPAQLQPGDCPPLELKNTIIEDGDMVDTGYGAMDFSTLQDTKCEVPLDIC |
| | | QSICKYPDYLQMSADPYGDSMFFCLRREQLFARHFWNRAGVMGDTVPT |
| | | DLYIKGTSANMRETPGSCVYSPSPSGSIITSDSQLFNKPYWLHKAQGHNN |
| | | GICWHNQLFVTVVDTTRSTNLTLCASTQNPVPSTYDPTKFKQYSRHVEEY |
| | | DLQFIFQLCTITLTAEVMSYIHSMNSSILENWNFGVPPPPTTSLVDTYRFVQ |
| | | SVAVTCQKDTTPPEKQDPYDKLKFWTVDLKEKFSSDLDQYPLGRKFLVQA |
| | | GLRRRPTIGPRKRPAASTSTASTASRPAKRVRIRSKK |
| 9 | Major Capsid | MVQILFYILVIFYYVAGVNVFHIFLQMSVWRPSEATVYLPPVPVSKWSTDE |
| | (L1) Protein | YVSRTSIYYYAGSSRLLTVGHPYFSIKNTSSGNGKKVLVPKVSGLQYRVFR |
| | of HPV type | IKLPDPNKFGFPDTSFYNPETQRLVWACTGLEIGRGQPLGVGISGHPLLNK |
| | 52 | FDDTETSNKYAGKPGIDNRECLSMDYKQTQLCILGCKPPIGEHWGKGTPC |
| | | NNNSGNPGDCPPLQLINSVIQDGDMVDTGFGCMDFNTLQASKSDVPIDIC |
| | | SSVCKYPDYLQMASEPYGDSLFFFLRREQMFVRHFFNRAGTLGDPVPGD |
| | | LYIQGSNSGNTATVQSSAFFPTPSGSMVTSESQLFNKPYWLQRAQGHNN |
| | | GICWGNQLFVTVVDTTRSTNMTLCAEVKKESTYKNENFKEYLRHGEEFDL |
| | | QFIFQLCKITLTADVMTYIHKMDATILEDWQFGLTPPPSASLEDTYRFVTST |
| | | AITCQKNTPPKGKEDPLKDYMFWEVDLKEKFSADLDQFPLGRKFLLQAGL |
| | | QARPKLKRPASSAPRTSTKKKKVKR |
| 10 | Major Capsid | MVLILCCTLAILFCVADVNVFHIFLQMSVWRPSEATVYLPPVPVSKVVSTDE |
| | (L1) Protein | YVSRTSIYYYAGSSRLLAVGNPYFSIKSPNNNKKVLVPKVSGLQYRVFRVR |
| | of HPV type | LPDPNKFGFPDTSFYNPDTQRLVWACVGLEIGRGQPLGVGVSGHPYLNK |
| | 58 | FDDTETSNRYPAQPGSDNRECLSMDYKQTQLCLIGCKPPTGEHWGKGVA |
| | | CNNNAAATDCPPLELFNSIIEDGDMVDTGFGCMDFGTLQANKSDVPIDICN |
| | | STCKYPDYLKMASEPYGDSLFFFLRREQMFVRHFFNRAGKLGEAVPDDL |
| | | YIKGSGNTAVIQSSAFFPTPSGSIVTSESQLFNKPYWLQRAQGHNNGICW |
| | | GNQLFVTVVDTTRSTNMTLCTEVTKEGTYKNDNFKEYVRHVEEYDLQFVF |
| | | QLCKITLTAEIMTYIHTMDSNILEDWQFGLTPPPSASLQDTYRFVTSQAITC |
| | | QKTAPPKEKEDPLNKYTFWEVNLKEKFSADLDQFPLGRKFLLQSGLKAKP |
| | | RLKRSAPTTRAPSTKRKKVKK |
| 11 | Latent | MEHDLERGPPGPRRPPRGPPLSSSLGLALLLLLLALLFWLYIVMSDWTGGALL |
| | membrane | VLYSFALMLIIIILIIFIFRRDLLCPLGALCILLLMITLLLIALWNLHGQAL |
| | protein 1 | FLGIVLFIFGCLLVLGIWIYLLEMLWRLGATIWQLLAFFLAFFLDLILLIIA |
| | (LMP1) | LYLQQNWWTLLVDLLWLLLFLAILIWMYYHGQRHSDEHHHDDSLPHPQQATD |
| | | DSGHESDSNSNEGRHHLLVSGAGDGPPLCSQNLGAPGGGPDNGPQDPDNT |
| | | DDNGPQDPDNTDDNGPHDPLPQDPDNTDDNGPQDPDNTDDNGPHDPL |
| | | PHSPSDSAGNDGGPPQLTEEVENKGGDQGPPLMTDGGGGHSHDSGHG |
| | | GGDPHLPTLLLGSSGSGGDDDDPHGPVQLSYYD |

The present invention is not limited to the aforementioned viral proteins. For example, the present invention also includes variants of the aforementioned viral proteins.

In some embodiments, the compositions described herein (e.g., Sarravis® Core) may be combined with viral proteins. In some embodiments, the viral proteins comprise the aforementioned sequences (e.g., SEQ ID NO: 1 to SEQ ID NO: 11) but are not limited to those sequences. In some embodiments, the compositions described herein (e.g., Sarravis® Core) may be combined with viral proteins comprising 100% homology with the aforementioned viral proteins. In other embodiments, the compositions described herein (e.g., Sarravis® Core) may be combined with viral proteins comprising about 95% homology with the aforementioned viral proteins. In other embodiments, the compositions described herein (e.g., Sarravis® Core) may be combined with viral proteins comprising about 90% homology with the aforementioned viral proteins. In some embodiments, the compositions described herein (e.g., Sarravis® Core) may be combined with viral proteins comprising about 85% homology with the aforementioned viral proteins. In other embodiments, the compositions described herein (e.g., Sarravis® Core) may be combined with viral proteins comprising about 75% homology with the aforementioned viral proteins. In some embodiments, the compositions described herein (e.g., Sarravis® Core) may be combined with viral proteins comprising about 50% homology with the aforementioned viral proteins.

In some embodiments, the compositions described herein (e.g., Sarravis® Core) may be necessary to upregulate RNAi genes such that vaccines may be rendered more effective for not just illness prevention, but also that of disease expression.

In some embodiments, the present invention may also feature a composition for enhancing gene silencing. In some embodiments, the composition comprises keratin and glutathione. In other embodiments, the present invention may feature a composition for enhancing p53 gene expression, the composition comprising keratin and magnesium and/or glutathione. In further embodiments, the present invention may feature a composition for enhancing AGO and/or DICER gene expression, the composition comprising keratin and magnesium and/or glutathione. In other embodiments, the composition comprises keratin and one or more cofactors. In some embodiments, the present invention may feature a composition of enhancing p53, AGO, and/or DICER effects. In some embodiments, the composition comprises keratin and magnesium and/or glutathione.

In other embodiments, the present invention may feature a method of enhancing gene silencing in a subject in need thereof. In some embodiments, the method comprises administering a therapeutic amount of any one of the compositions described herein, to the subject. In further embodiments, the present invention may also feature a method of enhancing the expression of p53 in a subject in need thereof. In some embodiments, the method comprises administering a therapeutic amount of any one of the compositions described herein to the subject.

In some embodiments, the present invention features a method of decreasing viral and/or cancer cell expression in a subject in need thereof. In some embodiments, the method comprises administering a therapeutic amount of any one of the compositions described herein, to the subject.

In some embodiments, the viral load is detected via a polymerase chain reaction (PCR). In other embodiments, the viral load is detected via mRNA ID testing for viruses. For example, the method of decreasing viral and/or cancer cell expression may further comprise testing the subject using a PCR test or mRNA ID test for a virus, administering the composition to the subject, and then testing the subject using a PCR test or mRNA ID test for the virus.

In some embodiments, the present invention features a method of treating a disease caused by a virus in a subject in need thereof. In some embodiments, the method comprises administering a therapeutic amount of any one of the compositions described herein, to the subject. In other embodiments, the method of treating a disease caused by a virus may further comprise testing the subject using a PCR test or mRNA ID test for a virus, administering the composition to the subject, and then testing the subject using a PCR test or mRNA ID test for the virus.

In conjunction with the embodiments described herein, the methods may further comprise administering a viral protein (see Table 1 for examples) combined with the composition described herein to the subject.

In some embodiments, the virus is severe acute respiratory syndrome coronavirus 2 (SARS-CoV-2). In some embodiments, the disease is Coronavirus Disease 2019 (COVID-19). In some embodiments, the viral protein is the coronavirus spike protein. In some embodiments, the spike protein comprises one or more subunits. In some embodiments, the one or more subunits of the coronavirus spike protein comprise a spike protein subunit 1 (S1), a spike protein subunit 2 (S2), or a combination thereof.

In other embodiments, the virus is human papillomavirus (HPV). In other embodiments, the disease is cervical cancer. In other embodiments, the viral protein is a major capsid protein (L1).

In some embodiments, the virus is herpes simplex virus (HSV) and the disease is oral and/or genital herpes. In some embodiments, the viral protein comprises subunits for HSV.

In some other embodiments, the virus is Epstein-Barr virus (EBV) and the disease is infectious mononucleosis. In further embodiments, the virus is human immunodeficiency virus (HIV) and the disease is AIDS.

For example, in one embodiment, the present invention features a method of treating an EBV infection in a subject in need thereof. The method may comprise administering a therapeutic amount of a composition comprising at least Sarravis® Core, to the subject.

In another embodiment, the present invention features a method of treating human papillomavirus (HPV) in a subject in need thereof. The method comprises administering a therapeutic amount of a composition comprising Sarravis® Core and valacyclovir, to the subject. In other embodiments, the method may further comprise testing the subject using a PCR test or mRNA ID test for a virus, administering the composition to the subject, and then testing the subject using a PCR test or mRNA ID test for the virus.

In yet another embodiment, the present invention features a method of treating a herpes simplex virus (HSV) in a subject in need thereof. The method comprises administering a therapeutic amount of a composition comprising Sarravis® Core, valacyclovir, and a protease inhibitor, to the subject. In yet another embodiment, the HSV treatment method may comprise administering a therapeutic amount of a composition comprising Sarravis® Core, DNA polymerase inhibitor, and a protease inhibitor, to the subject. In other embodiments, the method may further comprise testing the subject using a PCR test or mRNA ID test for a virus, administering the composition to the subject, and then testing the subject using a PCR test or mRNA ID test for the virus.

In some other embodiments, the present invention features a method of treating a bacterial infection. The method may comprise administering a therapeutic amount of a composition comprising Sarravis® Core. In some embodiments, the composition may further comprise herbs, antibiotics, or a combination thereof. Non-limiting examples of antibiotics include amoxicillin, cephalexin, and azithromycin. In some embodiments, the method may be used to treat bacterial vaginosis, strep throat, bacterial pneumonia, or urinary tract infections.

In some embodiments, compositions described herein are orally administered at a dose ranging from about 0.0001 g to 0.05 g. In some embodiments, compositions described herein are orally administered at a dose ranging from about 0.0001 g to 0.02 g, or about 0.0001 g to 0.01 g, or about 0.0001 g to 0.005 g, or about 0.0001 g to 0.0025 g, or about 0.0001 g to 0.001 g, or about 0.001 g to 0.02 g, or about 0.001 g to 0.01 g, or about 0.001 g to 0.005 g, or about 0.001 g to 0.0025 g, or about 0.0025 g to 0.02 g, or about 0.0025 g to 0.01 g, or about 0.0025 g to 0.005 g, or about 0.005 g to 0.02 g, or about 0.005 g to 0.01 g, or about 0.01 g to 0.02 g.

In some embodiments, compositions described herein are administered via a subcutaneous injection at a dose ranging from about 1 cubic centimeter (cc) to 10 cc, or about 1 cc to 8 cc or about 1 cc to 6 cc, or about 1 cc to 4 cc, or about 1 cc to 2 cc, or about 2 cc to 10 cc, or about 2 cc to 8 cc, or about 2 cc to 6 cc, or about 2 cc to 4 cc, or about 4 cc to 10 cc, or about 4 cc to 8 cc or about 4 cc to 6 cc, or about 6 cc to 10 cc, or about 6 cc to 8 cc, or about 8 cc to 10 cc.

In other embodiments, the composition described herein is administered via an intravenous injection using an IV solution (e.g., a saline solution). For example, a dose ranging from 1 cc to 10 cc of the composition may be added to 250 cc of an IV solution, or a dose ranging from 1 cc to 10 cc of the composition may be added to 50 cc of an IV solution, a dose ranging from 1 cc to 10 cc of the composition may be added to 500 cc of an IV solution. A non-limiting embodiment of an IV solution may comprise pitcher plant extracts and magnesium and/or glutathione in a saline solution. Another non-limiting embodiment of an IV solution may comprise keratin and magnesium and/or glutathione in a saline solution. The IV solutions as described herein are not meant to be limiting in any way and are only used to describe alternative ways the compositions described herein are delivered to a subject.

In other embodiments, compositions described herein are administered via an intravenous injection at a dose ranging from about 1 mL/40 kg to 10 mL/40 kg, or from about 1 mL/40 kg to 8 mL/40 kg, or from about 1 mL/40 kg to 6 mL/40 kg, or from about 1 mL/40 kg to 4 mL/40 kg, or from about 1 mL/40 kg to 2 mL/40 kg, or from about 2 mL/40 kg to 10 mL/40 kg, or from about 2 mL/40 kg to 8 mL/40 kg, or from about 2 mL/40 kg to 6 mL/40 kg, or from about 2 mL/40 kg to 4 mL/40 kg, or from about 4 mL/40 kg to 10 mL/40 kg, or from about 4 mL/40 kg to 8 mL/40 kg, or from about 4 mL/40 kg to 6 mL/40 kg, or from about 6 mL/40 kg to 10 mL/40 kg, or from about 6 mL/40 kg to 8 mL/40 kg, or from about 8 mL/40 kg to 10 mL/40 kg.

In some embodiments, compositions described herein may be administered once daily or twice daily. In another embodiment, compositions described herein may be administered at least once to four times daily. In some embodiments, compositions described herein may be administered at least once daily, at least once every other day, at least once weekly, or once, twice, or 3 times per week.

In some embodiments, compositions described herein are administered orally. In other embodiments, compositions described herein are administered sublingually. In further embodiments, compositions described herein are administered parenterally (e.g., intravenously and subcutaneously). In some embodiments, compositions described herein are administered by intramuscular injection (i.e., intramuscularly). In other embodiments, compositions described herein are administered topically (including vaginally (e.g., paravaginally), or rectally (e.g., pararectally).

In some embodiments, the composition is formulated for oral administration, topical administration, or intramuscular, subcutaneous or intravenous injections. In another embodiment, the composition may be formulated with sterile water to produce an injectable medicine. In yet another embodiment, the composition is in the form of a lotion, cream, oil, balm, gel, injectable solution, or oral tincture. In some embodiments, the composition is formulated as a lozenge (i.e., as a solid preparation intended to dissolve or disintegrate slowly in the mouth) for a sublingual medicine. In other embodiments, the composition is formulated into a vaginal suppository for use paravaginally.

In some embodiments, the present invention features a method of enhancing viral load detection in a subject, the method comprising administering Sarravis® Core to the subject. In other embodiments, the present invention may feature a method of enhancing viral load identification in a subject, the method comprising administering a composition comprising 0.1%-99% vol and/or % wt of keratin and 0.01%-95% vol and/or % wt of one or more cofactors, to the subject.

In some embodiments, the viral load is detected via a polymerase chain reaction (PCR). In other embodiments, the viral load is detected via mRNA ID testing for viruses. For example, the method to better ID viral load/presence may comprise performing a PCR test or mRNA ID test for a virus, administering a composition of the present invention, and then performing another PCR test or mRNA ID test.

Without wishing to limit the present invention to any theories or mechanisms it is believed that the compositions described herein (e.g., Sarravis® Core) causes a rise in RNAi cofactors (e.g., DICER and AGO2) which then causes the release of integrated viral particles and abnormal cell load. This allows for better detection of the virus, and therefore a better measurement and treatment of viral load in a subject. For example, a rise in DICER and AGO2 mRNA caused an appearance of viral load in tissues previously diagnosed as within normal limits (see Example 1).

### EXAMPLES

The following are non-limiting examples of the present invention. It is to be understood that said examples are not intended to limit the present invention in any way. Equivalents or substitutes are within the scope of the present invention.

### Example 1

Sarravis® Core with magnesium was used for treatment of a chronic HPV infection in a female patient. Dosing of said medicament was administered paravaginally at 10 mg once daily for one month. The patient experienced pelvic pain and vaginal bloody discharge within 24 hours of the first dose. Symptoms remained for one week as such, with pelvic pain diminishing first as time went on through the month. Vaginal discharge (of blood and later mucus and clots) remained at the time of the second sample (listed as “post” on Table 2).

Table 2 shows mRNA levels in the patient administered Sarravis® Core medicament. This patient took the therapy topically to labial tissues daily, over the course of two months.

| | p53 | AGO2 | DICER | HPV |
|---|---|---|---|---|
| | mRNA | mRNA | mRNA | mRNA |
| | levels | levels | levels | levels |
| Pre-Sarravis ® | 983,135 | 2,945 | 183 | 5,568 |
| Core | | | | |
| Post-Sarravis ® | 1,039,919 | 4,435 | 404 | 7,118 |
| Core | | | | |

The patient was later prescribed Sarravis® Core with valacyclovir (SarraV), which was administered paravaginally. The HPV infection cleared up.

### Example 2

A female patient had previously done chemo/radiation but she still had metastatic cervical cancer. She began a new treatment of Sarravis® Core with valacyclovir (SarraV) administered intravaginally

During March 2022, the patient underwent 3× weekly intravaginal insertion of SarraV. She experienced vaginal discharge and clots during that time. In April 2022, she used SarraV for two weeks and then stopped using the SarraV a week before her positron emission tomography (PET) scan. She went for her PET and the results demonstrated that she was cancer-free. SarraV was able to clear her metastatic cervical cancer. During May-June 2022, the patient used SarraV intermittently as an intravaginal treatment, 0-2× per week. Table 3A and 3B shows the gene panel (m RNA results) of the patient. When the patient went back to intermittent use of SarraV PV in 5-6/2022, the gene panel showed how the genes varied.

Table 3A shows a gene panel (mRNA levels) taken from a urine sample of the patient that was administered the SarraV medicament.

| | | Pre- | During | 2 Weeks of | Intermittent | |
|---|---|---|---|---|---|---|
| | | SarraV | SarraV | SarraV | SarraV | |
| | | February 2022 | March 2022 | April 2022 | May 2022 | June 2022 |
| | | urine | urine | urine | urine | urine |
| DICER | clips nonself genes | 5325 | 7277 | 1748 | 6485 | 6651 |
| NFKB | inflammation | 29771 | 43002 | 6918 | 39817 | 41870 |
| HPV | HPV | 3 | 1 | 27 | 0 | 17 |
| EBV | Epstein Barr | 67 | 258 | 636 | 91 | 647 |
| HSV | Herpes simplex | 89 | 277 | 336 | 146 | 314 |
| S 1/2 | Covid | 0 | 1 | 1022 | 2 | 596 |
| SOD | superoxide dismu. | 210569 | 308825 | 33318 | 166512 | 328614 |
| QSOX | sulfhydryl oxidase | 29779 | 42828 | 9003 | 39861 | 38998 |
| p53 | p53 | 75559 | 123872 | 17453 | 134826 | 94296 |
| TXNRD | Thioredoxin red. | 124859 | 175108 | 26135 | 164224 | 138366 |
| MMP2 | ECM remodeling | 24 | 38 | 89 | 15 | 164 |
| NEIL | DNA glycosylase | 39250 | 56498 | 9005 | 52953 | 51890 |
| NOX | EMT driver | 108664 | 178716 | 17010 | 191958 | 94340 |
| OGG1 | ox stress | 37948 | 52876 | 8026 | 51301 | 48513 |
| ATM | DNA repair | 912591 | 1249910 | 194642 | 1151790 | 1168935 |
| ATR | DNA repair | 27487 | 45482 | 6003 | 48353 | 35178 |
| BRCA | DNA repair | 157884 | 214759 | 33159 | 4129169 | 212363 |
| ERCC | DNA repair | 12530 | 23057 | 3209 | 24665 | 18335 |
| MLH | DNA repair | 52089 | 80557 | 12875 | 70223 | 78056 |
| MSH | DNA repair | 247417 | 376585 | 54594 | 355854 | 327958 |
| MUTYH | DNA repair | 12168 | 20828 | 3380 | 18777 | 19519 |
| MSH | DNA repair | 104125 | 140619 | 23172 | 120132 | 147404 |
| MT | DNA repair | 1055 | 2694 | 7485 | 15439 | 8587 |
| PO | DNA polymerase | 1309687 | 1801440 | 275861 | 1680660 | 1597204 |

Table 3B shows a gene panel (mRNA levels) taken from a vaginal sample of the patient that was administered the SarraV medicament.

| | | Pre- | During | 2 Weeks of | Intermittent | |
|---|---|---|---|---|---|---|
| | | SarraV | SarraV | SarraV | SarraV | |
| | | February 2022 | March 2022 | April 2022 | May 2022 | June 2022 |
| | | vaginal | vaginal | vaginal | vaginal | vaginal |
| DICER | clips nonself genes | 11060 | 22137 | 16879 | 18101 | 14982 |
| NFKB | inflammation | 52099 | 57922 | 75463 | 36702 | 52041 |
| HPV | HPV | 5 | 4 | 8 | 2 | 7 |
| EBV | Epstein Barr | 545 | 526 | 862 | 506 | 533 |
| HSV | Herpes simplex | 288 | 420 | 630 | 275 | 368 |
| S 1/2 | Covid | 6 | 5 | 15 | 2 | 2 |
| SOD | superoxide dismu. | 148277 | 284738 | 166431 | 74782 | 121268 |
| QSOX | sulfhydryl oxidase | 51159 | 29961 | 46698 | 21323 | 22763 |
| p53 | p53 | 10910 | 8705 | 23244 | 8438 | 12706 |
| TXNRD | Thioredoxin red. | 31097 | 47784 | 42033 | 29557 | 135167 |
| MMP2 | ECM remodeling | 470 | 242 | 548 | 584 | 1071 |
| NEIL | DNA glycosylase | 6613 | 4248 | 11752 | 3787 | 5561 |
| NOX | EMT driver | 10010 | 3912 | 17373 | 4087 | 7426 |
| OGG1 | ox stress | 5501 | 4109 | 9097 | 3157 | 4706 |
| ATM | DNA repair | 105818 | 62567 | 184317 | 56484 | 83727 |
| ATR | DNA repair | 3694 | 3166 | 6569 | 2779 | 4232 |
| BRCA | DNA repair | 23911 | 16079 | 42122 | 14944 | 21300 |
| ERCC | DNA repair | 3823 | 2599 | 6455 | 2301 | 3590 |
| MLH | DNA repair | 15545 | 14611 | 27447 | 12479 | 16585 |
| MSH | DNA repair | 34358 | 22876 | 56669 | 20153 | 27785 |
| MUTYH | DNA repair | 4258 | 3075 | 8242 | 3199 | 4616 |
| MSH | DNA repair | 15847 | 9998 | 29231 | 8849 | 13212 |
| MT | DNA repair | 154647 | 95042 | 247697 | 93243 | 130860 |
| PO | DNA polymerase | 272122 | 215081 | 453588 | 171668 | 211982 |

### Example 3

The use of Sarravis® Core (i.e., a composition described herein) with glutathione, magnesium, and riboflavin to stabilize metastatic prostate cancer.

Table 4 demonstrates pre and post use of a Sarravis® Core (with magnesium) medicament used topically (containing magnesium) at 10 mg per dosing and via IV administration (containing glutathione and magnesium) at 1 mL/250 cc normal saline IV in a patient with metastatic prostate cancer. This patient was on the therapy for a year prior to the taking of MRI results. Initial symptoms with use included “the worst” experience of body aches and fever the patient had ever experienced previously. Symptoms resolved within 48 hours completely and never returned with subsequent medicament administration.

| | Diagnosis (per MRI) | Lab Findings |
|---|---|---|
| Pre-Sarravis ® | Metastatic prostate cancer | prostate-specific |
| Core | | antigen |
| | | (PSA) = 17.6 |
| | | in August 2019 |
| Post-Sarravis ® | Stable metastatic prostate | PSA = 15.5 |
| Core | cancer (i.e., no evidence of | in January 2011 |
| | further spread of cancer) | |

### Example 4: Long COVID Treatment

A COVID long-haul patient had COVID on and off for about 1 year. The patient was administered SarraV (Sarravis® Core with valacyclovir) plus a protease inhibitor. The patient's COVID symptoms went away and the patient was successfully treated.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A composition comprising about 0.1%-99% wt of keratin, and about 0.01%-95% wt of magnesium or glutathione, or both magnesium and glutathione.

2. The composition of claim 1, wherein the keratin is extracted from pitcher plant or produced synthetically.

3. The composition of claim 1, further comprising zinc, chromium, selenium, B-vitamins, cannabidiol, tetrahydrocannabinol, or a combination thereof.

4. The composition of claim 1, further comprising a drug product, wherein the drug product comprises an antiviral drug, an anti-inflammatory drug, a cancer-modulating drug, an immune-modulating drug, a DNA/RNA polymerase inhibitor, a protease inhibitor, or a combination thereof.

5. The composition of claim 4, wherein the drug product comprises acyclovir, valacyclovir, vidarabine, famciclovir, nitazoxanide, remdesivir, nirmatrelvir, ritonavir, molnupiravir, zanamivir, peramivir, baloxavir marboxil, oseltamivir phosphate, saquinavir, indinavir, nelfinavir, amprenavir, fosamprenavir, lopinavir, atazanavir, tipranavir, darunavir, asunaprevir, boceprevir, grazoprevir, glecaprevir, paritaprevir, simeprevir, telaprevir, ensitrelvir, cisplatin, mercaptopurine, carboplatin, paclitaxel, topotecan, vinblastine, gemcitabine docetaxel, doxorubicin, α-amanitin, mithramycin A, tenofovir, abacavir hemisulfate, delavirdine mesylate, azidothymidine, aphidicolin, entecavir, favipiravir, galidesivir dihydrochloride, BMS 986094, EIDD 1931, GS 441524, BMH 21, emetine dihydrochloride, rilpivirine, acetaminophen, cortisone, hydrocortisone, prednisone, indomethacin, ibuprofen, aspirin, celecoxib, or naproxen, or a combination thereof.

6. The composition of claim 1, wherein the composition is effective for decreasing viral and/or cancer cell expression, enhancing gene silencing, enhancing p53 gene expression, upregulating RNA interference, or a combination thereof.

7. The composition of claim 1, wherein the composition is formulated for oral, sublingual, or topical administration, or for intramuscular, subcutaneous or intravenous injections.

8. A composition comprising:
a. about 0.1%-99% wt of an herbal preparation of a carnivorous plant;
b. about 0.01%-95% wt of magnesium, glutathione, or both; and
c. about 0.01%-95% wt of a drug product.

9. The composition of claim 8, wherein the herbal preparation of the carnivorous plant is prepared from Sarracenia flava, Sarracenia purpurea, or mixed hybrids thereof.

10. The composition of claim 8, further comprising zinc, chromium, selenium, B-vitamins, keratin, cannabidiol, tetrahydrocannabinol, or a combination thereof.

11. The composition of claim 8, wherein the drug product comprises an antiviral drug, an anti-inflammatory drug, a cancer-modulating drug, an immune-modulating drug, a polymerase inhibitor, a protease inhibitor, or a combination thereof.

12. The composition of claim 8, wherein the drug product comprises acyclovir, valacyclovir, vidarabine, famciclovir, nitazoxanide, remdesivir, nirmatrelvir, ritonavir, molnupiravir, zanamivir, peramivir, baloxavir marboxil, oseltamivir phosphate, saquinavir, indinavir, nelfinavir, amprenavir, fosamprenavir, lopinavir, atazanavir, tipranavir, darunavir, asunaprevir, boceprevir, grazoprevir, glecaprevir, paritaprevir, simeprevir, telaprevir, ensitrelvir, cisplatin, mercaptopurine, carboplatin, paclitaxel, topotecan, vinblastine, gemcitabine docetaxel, doxorubicin, α-amanitin, mithramycin A, tenofovir, abacavir hemisulfate, delavirdine mesylate, azidothymidine, aphidicolin, entecavir, favipiravir, galidesivir dihydrochloride, BMS 986094, EIDD 1931, GS 441524, BMH 21, emetine dihydrochloride, rilpivirine, acetaminophen, cortisone, hydrocortisone, prednisone, indomethacin, ibuprofen, aspirin, celecoxib, or naproxen, or a combination thereof.

13. The composition of claim 8, wherein the composition is effective for decreasing viral and/or cancer cell expression, enhancing gene silencing, enhancing p53 gene expression, upregulating RNA interference, or a combination thereof.

14. The composition of claim 8, wherein the composition is formulated for oral or topical administration, or for intramuscular, subcutaneous or intravenous injections.

15. A method of enhancing gene silencing in a subject in need thereof, the method comprising administering to the subject a therapeutic amount of a composition according to claim 8.

16. A method of treating a viral disease in a subject in need thereof, the method comprising administering to the subject a therapeutically effective amount of a composition according to claim 8.

17. The method of claim 16, further comprising:
a. prior to administering the composition, testing the subject for a virus using polymerase chain reaction (PCR) or mRNA testing, and
b. after administering the composition, retesting the subject for the virus using PCR or mRNA testing, wherein if the virus is still present in the subject, a result of the testing will come back positive and/or will indicate presence of mRNA for that particular virus.

18. The method of claim 16, wherein the viral disease is Coronavirus Disease 2019 (COVID-19), a human papillomavirus (HPV) infection, a herpes simplex virus (HSV) infection, or mononucleosis caused by Epstein-Barr virus (EBV).

19. A composition for treating a human papillomavirus (HPV) or herpes simplex virus (HSV) infection, said composition comprises about 0.1%-99% wt of an herbal preparation of a pitcher plant, about 0.01%-95% wt of magnesium, and about 0.01%-95% wt of valacyclovir.

20. The composition of claim 19, further comprising about 0.01%-95% wt of a protease inhibitor, a DNA/RNA polymerase inhibitor, or a combination thereof.


SEQUENCE LISTING (11 sequences)

SEQ ID NO: 1 - protein, 1273 aa, Coronavirus
  REGION 1..1273: note = Severe acute respiratory syndrome coronavirus 2 (SARS-CoV-2)Spike Protein
  SIGNAL 1..14: note = signal sequence
  REGION 15..685: note = S1 subunit
  REGION 686..1273: note = S2 subunit
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 505 aa, Human papillomavirus type 16
  REGION 1..505: note = Major Capsid (L1) Protein
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 3 - protein, 568 aa, Human papillomavirus type 18
  REGION 1..568: note = Major Capsid (L1) Protein
MCLYTRVLILHYHLLPLYGPLYHPRPLPLHSILVYMVHIIICGHYIILFLRNVNVFPIFLQMALWRPSDNTVYLPPPSVARVVNTDDYVTPTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMPASPGSCVYSPSPSGSIVTSDSQLFNKPYWLHKAQGHNNGVCWHNQLFVTVVDTTPSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 4 - protein, 500 aa, Human papillomavirus type 6
  REGION 1..500: note = Major Capsid (L1) Protein
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVATSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYRGRSSIRTGVKRPAVSKASAAPKRKRAKTKR

SEQ ID NO: 5 - protein, 501 aa, Human papillomavirus type 11
  REGION 1..501: note = Major Capsid (L1) Protein
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 6 - protein, 504 aa, Human papillomavirus type 31
  REGION 1..504: note = Major Capsid (L1) Protein
MSLWRPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKTAPQKPKEDPFKDYVFWEVNLKEKFSADLDQFPLGRKFLLQAGYRARPKFKAGKRSAPSASTTTPAKRKKTKK

SEQ ID NO: 7 - protein, 499 aa, Human papillomavirus type 33
  REGION 1..499: note = Major Capsid (L1) Protein
MSVWRPSEATVYLPPVPVSKVVSTDEYVSRTSIYYYAGSSRLLAVGHPYFSIKNPTNAKKLLVPKVSGLQYRVFRVRLPDPNKFGFPDTSFYNPDTQRLVWACVGLEIGRGQPLGVGISGHPLLNKFDDTETGNKYPGQPGADNRECLSMDYKQTQLCLLGCKPPTGEHWGKGVACTNAAPANDCPPLELINTIIEDGDMVDTGFGCMDFKTLQANKSDVPIDICGSTCKYPDYLKMTSEPYGDSLFFFLRREQMFVRHFFNRAGTLGEAVPDDLYIKGSGTTASIQSSAFFPTPSGSMVTSESQLFNKPYWLQRAQGHNNGICWGNQVFVTVVDTTRSTNMTLCTQVTSDSTYKNENFKEYIRHVEEYDLQFVFQLCKVTLTAEVMTYIHAMNPDILEDWQFGLTPPPSASLQDTYRFVTSQAITCQKTVPPKEKEDPLGKYTFWEVDLKEKFSADLDQFPLGRKFLLQAGLKAKPKLKRAAPTSTRTSSAKRKKVKK

SEQ ID NO: 8 - protein, 539 aa, Human papillomavirus type 45
  REGION 1..539: note = Major Capsid (L1) Protein
MAHNIIYGHGIIIFLKNVNVFPIFLQMALWRPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 9 - protein, 529 aa, Human papillomavirus type 52
  REGION 1..529: note = Major Capsid (L1) Protein
MVQILFYILVIFYYVAGVNVFHIFLQMSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 10 - protein, 524 aa, Human papillomavirus type 58
  REGION 1..524: note = Major Capsid (L1) Protein
MVLILCCTLAILFCVADVNVFHIFLQMSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 11 - protein, 386 aa, Epstein Barr virus
  REGION 1..386: note = Latent membrane protein 1 (LMP1)
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD